(19) United States
(12) Patent Application Publication
(10) Pub. No.: US 2021/0085920 A1
(43) Pub. Date: Mar. 25, 2021

(54) STRAIN RELIEF AND METHODS OF USE THEREOF

(71) Applicant: Cook Medical Technologies LLC, Bloomington, IN (US)

(72) Inventors: Erin Roberts, Bloomington, IN (US); Elizabeth Brown, Bloomington, IN (US); Jorge L. Jimenez-Rios, Ph.D., Bloomington, IN (US); Lyle Hundley, Bloomington, IN (US); Johnny P. Smith, Worthington, IN (US); Jonathan Sheets, Bloomington, IN (US); Jaimie Jarboe, Bloomington, IN (US); Nathan Steinbrunner, Bloomington, IN (US); Cayley Gubser, Bloomington, IN (US); Jeffry S. Melsheimer, Springville, IN (US); Samantha Charley, Ft. Wright, KY (US); Tyler Dow, Martinsville, IN (US); Kathryn R. Hardert, Bloomington, IN (US); David Gordon, Bloomington, IN (US); Mitchell T. Aman, Canton, OH (US); Kristen M. Bunch, Bloomington, IN (US); Wesley Pedersen, Bloomington, IN (US); Shawn L. Nichols, Bloomington, IN (US)

(73) Assignee: Cook Medical Technologies LLC, Bloomington, IN (US)

(21) Appl. No.: 16/836,580

(22) Filed: Mar. 31, 2020

Related U.S. Application Data
(60) Provisional application No. 62/904,094, filed on Sep. 23, 2019.

(51) Int. Cl. A61M 25/00; B29C 45/14

(52) U.S. Cl. CPC A61M 25/0043; A61M 25/0009; B29L 2031/753; A61M 2025/0098; B29C 45/14336

(57) ABSTRACT

The present embodiments provide strain relief members for a medical device delivery system, methods of use, and methods of manufacturing. In one embodiment, a medical device delivery system may include a body member with a first end having a first outer diameter, a second end having a second outer diameter, an inner surface facing a lumen that extends axially through the body member along a longitudinal axis, and an outer surface opposite to the inner surface. The embodiment also includes a threaded first connector at the first end of the body member. The outer surface of the body member may include a plurality of depressions. The body member may include a first material. The first connector may include a second material. The first material is more flexible than the second material. A portion of the outer surface of the body member may cover a portion of the first connector.

### CROSS-REFERENCE TO RELATED APPLICATIONS

This patent application claims priority from U.S. Provisional Application No. 62/904,094, filed Sep. 23, 2019, the entirety of which is hereby fully incorporated by reference herein.

### TECHNICAL FIELD

This invention relates generally to medical devices, methods of use, and methods of manufacturing and more particularly, a strain relief member for a medical device delivery system.

### BACKGROUND

When in use, a medical device may enter a patient at a convenient insertion location and then be urged to a target region. Once the distal portion of the medical device has entered the patient, a physician may urge the distal tip forward by applying longitudinal forces to the proximal portion of the medical device. To effectively communicate these longitudinal forces, it may be desirable for at least a portion of the device to have a level of pushability and kink resistance, particularly near the proximal end.

The path taken by a medical device within a patient may be tortuous, requiring the medical device to change direction frequently. In some cases, it may even be necessary for the medical device to double back on itself. Movement within a patient may also require precision. While advancing a medical device during a procedure, a physician may apply torsional forces to the proximal portion of the device to aid in steering the device. Torsional forces applied on the proximal end may translate to the distal end to aid in steering. It may be desirable, therefore, that the proximal portion of a medical device have a level of torqueablility to facilitate steering.

To facilitate manipulation of the proximal end of the medical device and/or to interface with ancillary devices, medical devices may include a proximal hub or manifold. Such a hub may include a port or connector for connecting the medical device to a handle or other device. In some devices, hubs may be adhesively bonded to the device along with a tubular strain relief. Due at least in part to the way that a medical device is held or used during a medical procedure, however, known strain reliefs may not be designed to sufficiently prevent the device from bending, kinking, or separation, leading to a loss of device function. An improved strain relief is needed to protect at least a portion of the proximal end of a medical device, for example, at a handle junction.

### SUMMARY

The present disclosure provides a strain relief member for a medical device delivery system, methods of use thereof, and methods of manufacturing.

In one embodiment, a strain relief member for a medical device delivery system includes a body member having a first end with a first outer diameter, a second end with a second outer diameter, an inner surface facing a lumen that extends axially through the body member along a longitudinal axis, and an outer surface opposite to the inner surface. The outer surface of the body member includes a plurality of depressions. A threaded first connector is disposed at the first end of the body member. The body member includes a first material and the first connector includes a second material. The first material is more flexible than the second material. A portion of the outer surface of the body member covers at least a portion of the first connector.

In another embodiment, a strain relief member for a medical device delivery system includes a body member having a first end with a first outer diameter, a second end with a second outer diameter, an inner surface facing a lumen that extends axially through the body member along a longitudinal axis, and an outer surface opposite to the inner surface. The outer surface of the body member includes a plurality of depressions. A first connector is disposed at the first end of the body member. The body member includes a first material and the first connector includes a second material. The first material is more flexible than the second material. A medical device shaft is disposed through at least a portion of the lumen of the body member.

In a further embodiment, a method of manufacturing a strain relief member for a medical device delivery system includes injection molding a strain relief member. The strain relief member includes a body member having a first end with a first outer diameter, a second end with a second outer diameter, an inner surface facing a lumen that extends axially through the body member along a longitudinal axis, and an outer surface opposite to the inner surface. The outer surface of the body member comprises a plurality of depressions. A threaded first connector is disposed at the first end of the body member. The body member includes a first material and the first connector includes a second material. The first material is more flexible than the second material. A portion of the outer surface of the body member covers at least a portion of the first connector. A portion of the outer surface of the body member covers at least a portion of the first connector.

In a further embodiment, a method of manufacturing a strain relief member for a medical device delivery system includes injection molding a threaded first connector and over molding a strain relief member. The strain relief member includes a body member having a first end with a first outer diameter, a second end with a second outer diameter, an inner surface facing a lumen that extends axially through the body member along a longitudinal axis, and an outer surface opposite to the inner surface. The outer surface of the body member includes a plurality of depressions. The threaded first connector is disposed at the first end of the body member. The body member includes a first material and the first connector includes a second material. The first material is more flexible than the second material. A portion of the outer surface of the body member covers at least a portion of the first connector. A portion of the outer surface of the body member covers at least a portion of the first connector.

Other systems, methods, features and advantages will be, or will become, apparent to one with skill in the art upon examination of the following figures and detailed description. It is intended that all such additional systems, methods, features and advantages be within the scope of the disclosure, and be encompassed by the following claims.

### BRIEF DESCRIPTION OF THE DRAWINGS

The disclosure can be better understood with reference to the following drawings and description. The components in the figures are not necessarily to scale, emphasis instead being placed upon illustrating the principles of the disclosure. Moreover, in the figures, like referenced numerals designate corresponding parts throughout the different views. In certain instances, details may have been omitted that are not necessary for an understanding of aspects disclosed herein.

FIG. 1 is a sectional view of a first example of a strain relief member including a threaded connector.

FIG. 2 is a perspective view of the strain relief member of FIG. 1.

FIGS. 3A-3D are perspective views of four strain relief member embodiments, each including a plurality of depressions.

FIGS. 4A-4F are perspective views of six strain relief member embodiments with various outer depressions and diameters.

FIGS. 5A-5F are perspective views of six further strain relief member embodiments with various outer depressions and diameters.

FIG. 6 is a perspective view of a second example of a strain relief member including a threaded connector.

FIG. 7A is a perspective view of a handle, connector, and tube. FIGS. 7B-7C are perspective views of the device of FIG. 7A with the addition of strain relief body members and medical device shafts.

FIG. 8 is a sectional view of a third example of a strain relief member with a plurality of depressions.

FIG. 9 is a perspective view of the strain relief member of FIG. 8.

FIG. 10 is a sectional view of a first example of a coiled strain relief member including a connector.

FIG. 11 is a perspective view of a strain relief member coil component.

FIG. 12 is a perspective view of a second example of a coiled strain relief member including a connector.

FIG. 13 is a perspective view of a third example of a coiled strain relief member including a connector.

FIGS. 14A-14F are perspective views of various exemplary strain relief body member shapes, sizes, and depressions.

### DETAILED DESCRIPTION

In the present application, the term “proximal” refers to a direction that is generally towards a physician during a medical procedure, while the term “distal” refers to a direction that is generally towards a target site within a patient's anatomy during a medical procedure. Reference to coupling or connection of components, unless specified otherwise, includes direct connection as well as connection through intervening components. In this disclosure, reference is made to a physician. Reference to a physician includes any other suitable medical practitioners. For example, physician assistants, nurses, or other health professionals.

In general and as described in more detail herein with reference to reference numbers and Figures, a medical device 2 for introducing a tool 4 into a patient is shown and described herein. Such a tool 4 may include a retrieval device. The device 2 may be implemented for use with tools 4 configured to extend to a remote location within a patient but may be further or alternatively implemented for other clinical, diagnostic, observational or other medical uses such as, deployment of structure, interacting with tissue in a remote location, observation, and the like.

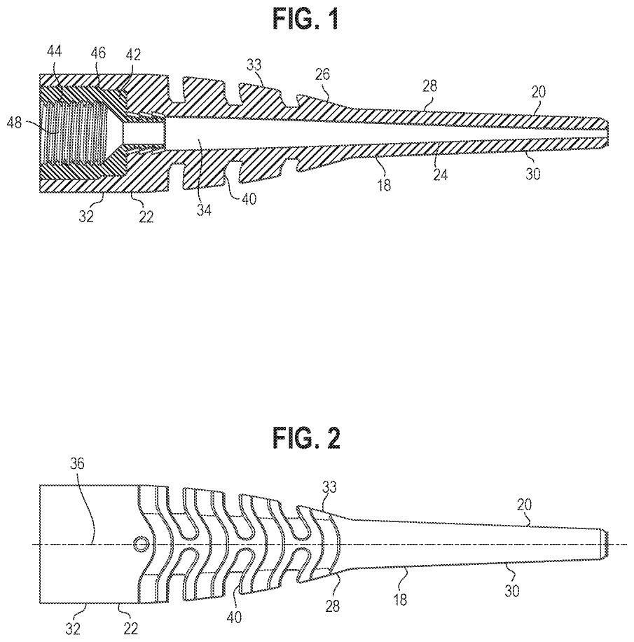

In some embodiments, as shown in FIGS. 1, 7B, and 7C, the medical device 2 may include a medical device shaft 6 and/or tube 38 with a tool 4 disposed at a distal end portion 8 of the medical device shaft 6 and a handle 12 connected to a proximal portion 10 of the medical device shaft 6. A medical device shaft 6 may be solid or hollow. The handle 12 may be a medical device 2 handle 12. The tool 4 may be a basket, while in other embodiments, the tool 4 may be a forceps, a snare, a loop, a laser fiber, an irrigation tube, the like, or a combination thereof.

A strain relief 18 may be connected to the proximal end portion 10 of the medical device shaft 6 and a proximal portion 22 of the strain relief 18 coupled with the distal end 14 of the handle 12. A strain relief 18 may include a pliable design. In some embodiments, the strain relief 18 provides support to the proximal portion 10 of the medical device shaft 6 and prevents at least a portion of the medical device shaft 6 from bending or kinking. A strain relief 18 may additionally or alternatively prevent separation at the junction of the medical device shaft 6 and the handle 12.

Referring to FIGS. 1 and 2, the strain relief 18 may include a body member 28 extending between a proximal end portion 32 and a distal end portion 30. The strain relief 18 may include a lumen 34 extending through the body member 28 between the proximal end portion 32 and the distal end portion 30 along a longitudinal axis 36. The lumen 34 may be open at the body member's 28 distal and proximal ends 30, 32, for example for insertion of a tube 38 or medical device shaft 6.

In some embodiments, multiple structures extend through and/or are disposed within the strain relief 18 lumen 34. As discussed above, a medical device shaft 6 may extend through the lumen 34. An inner tube 38 may extend through the lumen 34 over at least a portion of the surface of the medical device shaft 6. Example tubes 38 may include polymer tubing. FIG. 7A shows a combination of a handle 12, first connector 42, and a tube 38. Shown in FIG. 7B is the device of FIG. 7A with a strain relief 18 body member 28 covering at least a portion of the first connector 42 and a medical device shaft 6 disposed through the lumen 34 and tube 38. The tube 38 extends distally past a distal end 30 of the body member 28 of the strain relief 18. In another example, as shown in FIG. 7C, the device of FIG. 7A includes a strain relief 18 body member 28 covering at least a portion of the first connector 42 and a medical device shaft 6 disposed through the lumen 34 and tube 38, but the tube 38 is contained within the body member 28.

A strain relief 18 may additionally or alternatively include internal reinforcement, such as a nitinol cannula. Such internal reinforcement may contribute to prevention of kinking and separation from a handle 12 junction. An inner surface 24 of the strain relief 18 may face the lumen 34. An outer surface 26 of the strain relief 18 is located opposite to the inner surface 24.

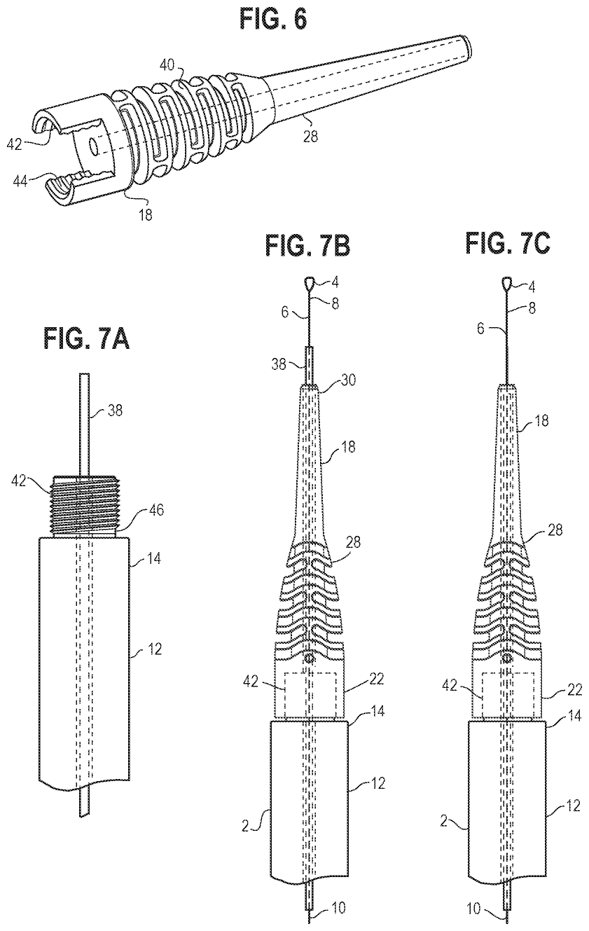

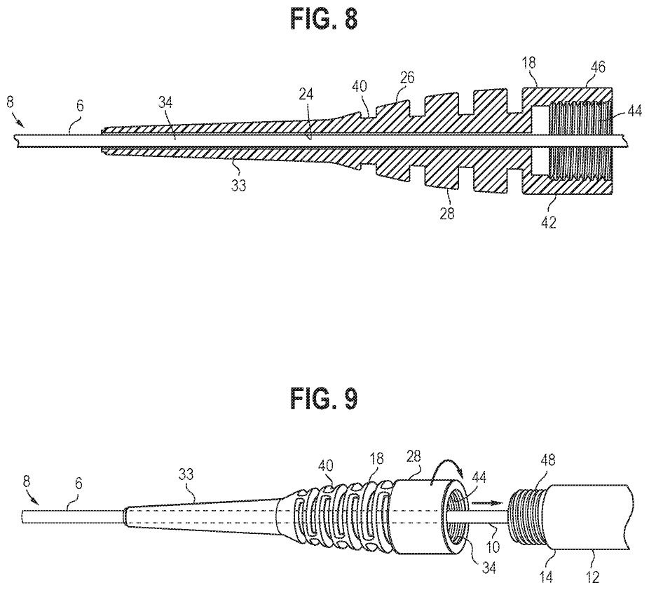

In some embodiments, as shown in FIGS. 1-9, the proximal end portion 22 and the distal end portion 20 of the strain relief 18 may each have a circular cross section to accommodate the configuration of a medical device shaft 6 that may be received within the strain relief 18. Additional configurations for the cross-sectional shape of the distal end and proximal end portions 20, 22 are possible including, but not limited to, oval, square, rectangular, triangular and combinations thereof.

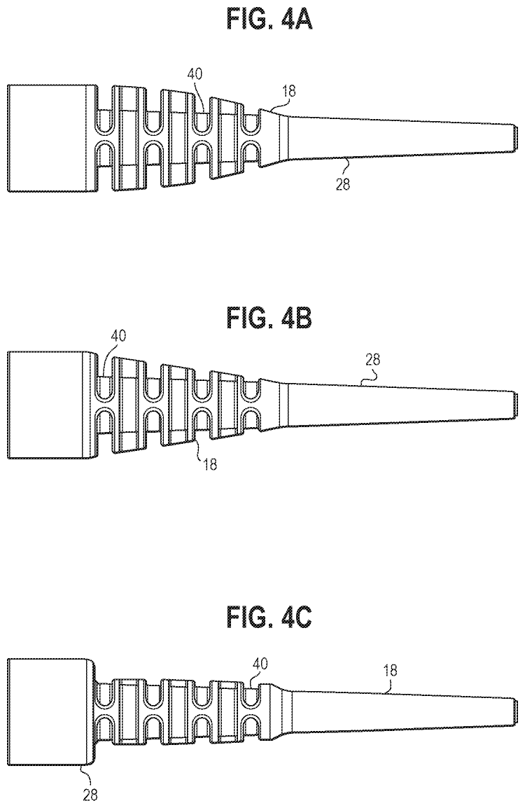

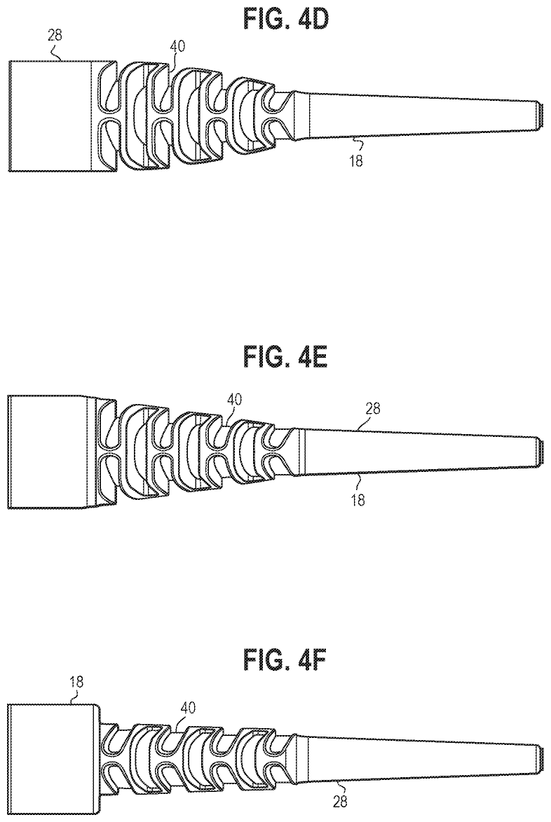

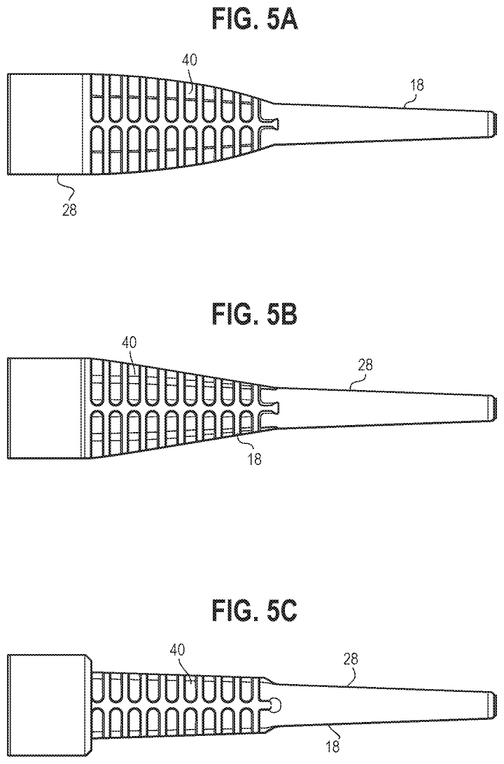

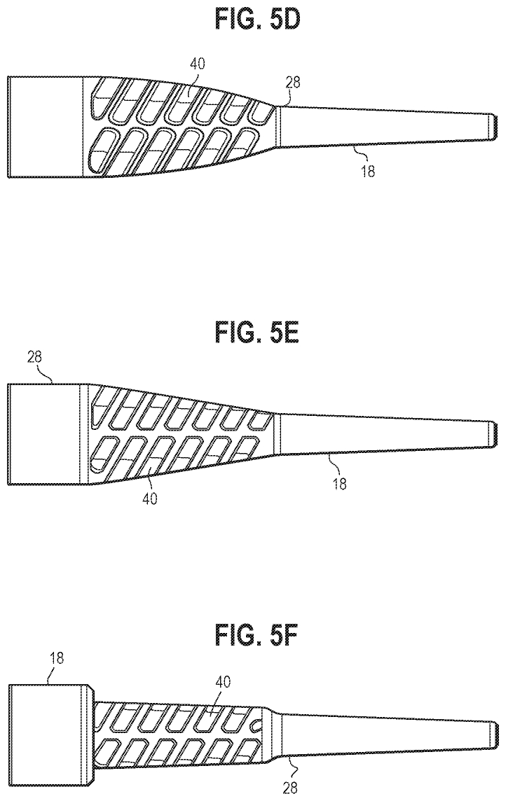

In some embodiments, one of the distal or proximal ends 20, 22 has a first outer diameter and the other one of the distal or proximal end 20, 22 has a second outer diameter. The outer diameter of the strain relief 18 may range from about 1.5 FR to about 25 FR. The strain relief 18 lumen 34 may have a diameter ranging from about 1.0 FR to about 8.0 FR. The term “about” as used in this specification is specifically defined to be a range that includes the reference value as well as plus or minus 5% of the reference value. For a tapered strain relief 18, the largest diameter may be approximately 0.40 inches/10 millimeters and taper down to a diameter larger than the outer diameter of component(s) disposed within the strain relief 18 lumen 34, such as a medical device shaft 6. The lumen 34 may have a diameter of approximately 8.0 FR/2.7 millimeters. The lumen 34 may have a consistent diameter throughout the body member 28, regardless of whether the outer surface 33 of the body member 28 is tapered or otherwise shaped. The value of a first outer diameter may be different than the value of the second outer diameter. For example, one such diameter may be larger than the other (as shown for example in FIGS. 1-9). FIGS. 4A-4C show an example pattern of offset depressions 40 but each of FIG. 4A, FIG. 4B, and FIG. 4C has different body member 28 diameters and depths of each depression 40. FIG. 4D, FIG. 4E, and FIG. 4F display another example pattern of depressions 40 using wavy lines and with different body member 28 diameters and depths of each depression 40. FIG. 5A, FIG. 5B, and FIG. 5C display another example pattern of depressions 40 using aligned, stacked lines and with different body member 28 diameters and depths of each depression 40. FIG. 5D, FIG. 5E, and FIG. 5F display another example pattern of depressions 40 using diagonal lines and with different body member 28 diameters and depths of each depression 40. The configuration (e.g., shape and dimension) of the cross sections of the distal end and proximal end portions 20, 22 may be varied as needed and/or desired, for example, to accommodate the configuration of various medical device shafts 6, handles 12, and/or other medical devices. The length of a strain relief 18 may be approximately 5 centimeters to 50 centimeters. However, dimensions of a strain relief 18 may be on scale with the sizing and needs of the particular medical device 2, including for example medical device 2 components such as a handle 12, medical device shaft 6, and tool 4. Such components that may need to be of a dimension to accommodate a particular medical procedure may be, for example, the strain relief 18 outer diameter(s), lumen 34 diameter, and/or strain relief 18 body member 28 length.

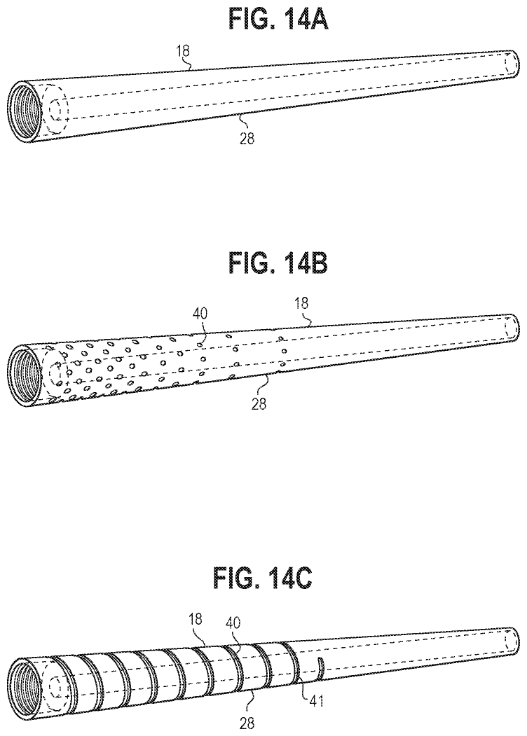

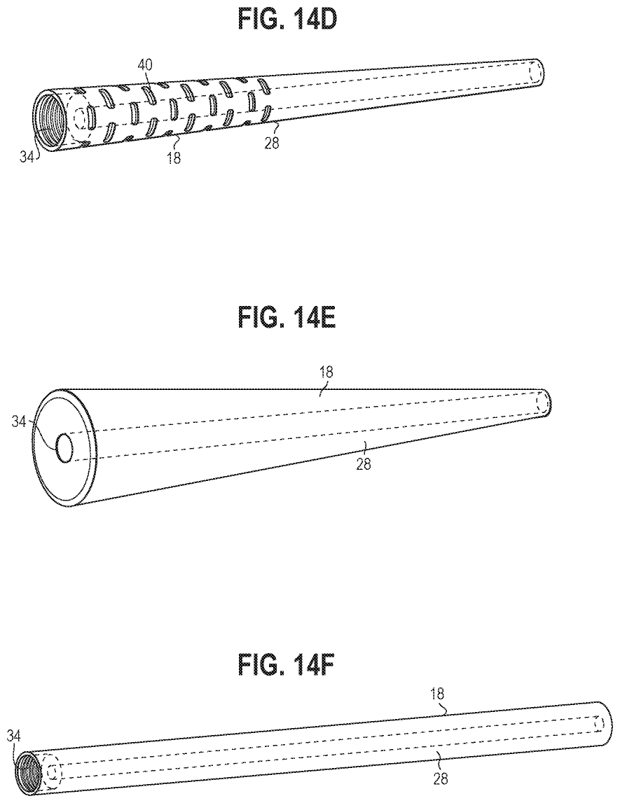

The strain relief 18 body member 28 may have a generally cylindrical or tubular shape with a substantially constant second outer diameter (e.g., FIG. 14F). In some embodiments, however, the proximal end portion 32 may taper in a distal direction between the proximal end portion 32 and the distal end portion 30 from a first, relatively larger, outer diameter to a second, relatively smaller, outer diameter. For example, as shown in FIG. 1, the body member 28 may have a funnel-shaped configuration. In some embodiments, the body member 28 may be conical, and may be a geometric conical profile with a constantly changing diameter along the length of the body member 28. In other embodiments, the proximal end portion 32 may be shaped similar to a geometric cone (i.e. with a decreasing diameter along its length, for example as shown in FIGS. 14A and 14E) but the rate of change of diameter may not be constant, such that a cross-section of the proximal end portion 32 forms a curve. The proximal end portion 32 may alternatively taper in a proximal direction between the proximal end portion 32 and the distal end portion 30 from a first, relatively larger, outer diameter to a second, relatively smaller, outer diameter.

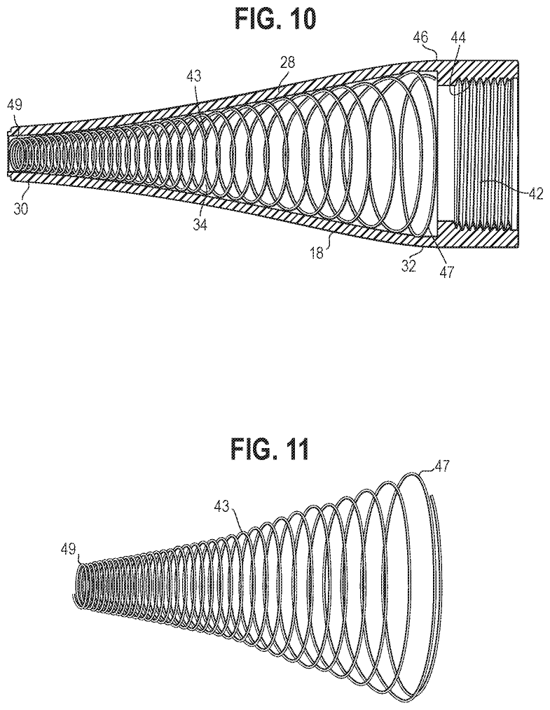

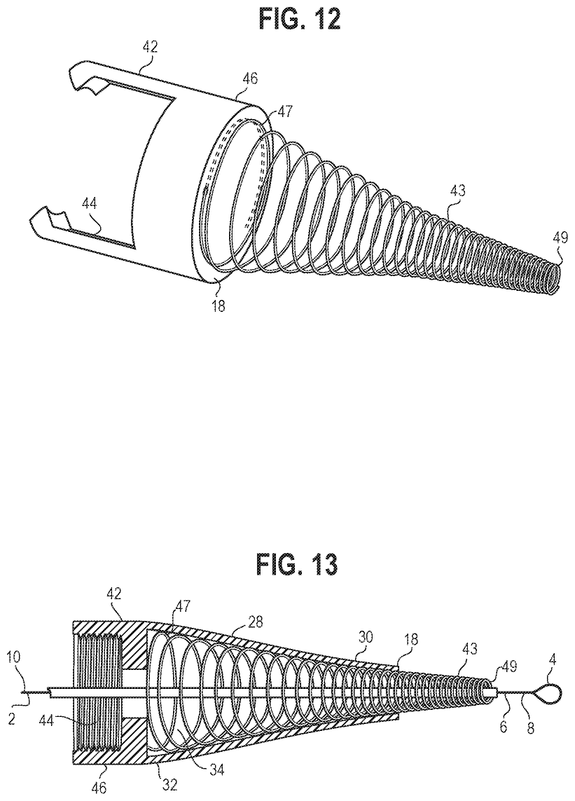

Referring to FIGS. 10-13, in some embodiments, the strain relief 18 may include a coil component 43 (e.g., a spring, as shown in FIG. 11). In some embodiments, the coil component 43 may extend through the lumen 34 of the strain relief 18 and may be entirely or partially contained within the lumen 34 (e.g., as shown in FIGS. 10 and 13). In some embodiments, the coil component 43 may be configured as the body member 28 of the strain relief 18 and may be connected to a connector 42 (e.g., as shown in FIG. 12). As shown in FIGS. 10-13, in some embodiments, the outer diameter of the coil component 43 may decrease along the length thereof in a distal direction between its proximal end portion 47 and its distal end portion 49. In some embodiments, as shown in FIGS. 10 and 13, the proximal end portion 47 of the coil component 43 is disposed closer to the proximal end portion 32 of the body member 28 than the distal end portion 49 of the coil component 43, with the distal end portion 49 of the coil component 43 disposed proximate the distal end portion 30 of the body member 28 within the lumen 34 (e.g., FIG. 10) or outside the lumen 34 (e.g., FIG. 13).

The strain relief 18 lumen 34 may be a tubular shape having a constant diameter. In some embodiments, however, as shown in FIG. 1, the strain relief 18 lumen 34 may also or alternatively taper in a distal direction between the proximal end portion 32 and the distal end portion 30 from a first, relatively larger, diameter to a second, relatively smaller, diameter. In some embodiments, the lumen 34 may be conical, and may be a geometric conical profile with a constantly changing diameter along the length of the body member 28. In other embodiments, the lumen 34 may be shaped similar to a geometric cone (i.e. with a decreasing diameter along its length) but the rate of change of diameter may not be constant, such that a cross-section of the lumen 34 forms a curve. The lumen 34 may alternatively taper in a proximal direction between the proximal end portion 32 and the distal end portion 30 from a first, relatively larger, outer diameter to a second, relatively smaller, outer diameter. The shape and size of a strain relief 18 lumen 34 may mimic the shape and/or size of a body member's 28 outer surface 33 and/or diameter(s) but the strain relief 18 lumen 34 scaled down in comparison in order to fit within the body member 28.

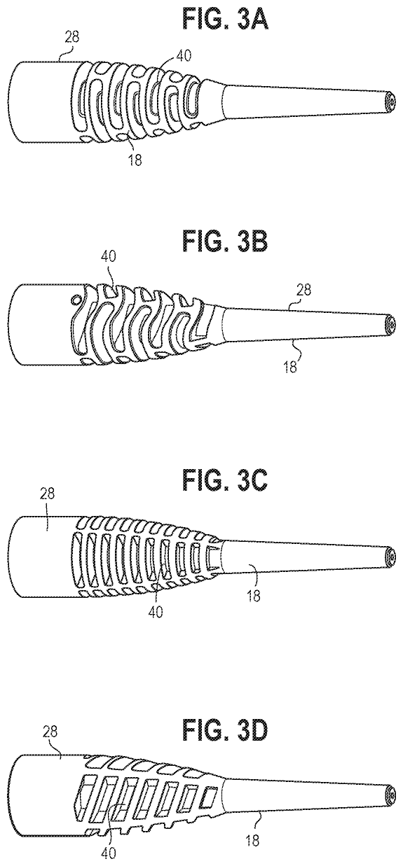

A strain relief 18 may include depressions or cut-outs 40 for additional flexibility and protection of a medical device shaft 6 and/or handle 12 junction during movement of a medical device 2. For example, as shown in FIGS. 1-6, 8-9, and 14B-14D, the strain relief 18 may include a depression or plurality of depressions 40, for example, on the outer surface 33 of the body member 28. Depressions 40 may be on only a portion of the body member 28, for example on approximately half the length of the body member 28 or on a central portion of the length of the body member 28. Alternatively, depressions 40 may be on the body member 28 spanning from the proximal end 32 to the distal end 30. In some examples, a depression 40 does not extend along the longitudinal axis 36, for example, such a depression 40 may be perpendicular to the longitudinal axis 36, as shown in FIGS. 3A and 3C, or diagonal to the longitudinal axis 36, as shown in FIG. 3D. A depression 40 may also or alternatively include a line shape, a wavy line shape (e.g., FIG. 3B), a circle and/or oval shape (e.g., FIGS. 14B and 14D), a coil 41 shape (e.g., 14C), or a combination thereof. In some examples (e.g., as shown in FIGS. 2-5) depressions 40 may be of similar shape but different sizes and/or lengths along the outer surface 33 of the body member 28. Depressions 40 may be made into the outer surface 33 of a body member 28, extending any suitable depth into the body member 28. As shown for example in FIGS. 1-5, depressions 40 may extend approximately half-way into the depth of the body member 28 between the outer surface 33 and the lumen 34. In some embodiments, different depressions 40 on the same body member 28 may extend into the body member 28 at various different depths. For example, a slight indentation may create a depression 40. A cut-out depression 40 may extend all the way or almost all the way through to the lumen 34 itself.

In some embodiments, the strain relief 18 may be extruded or injection molded. For example, the strain relief 18 may be formed as a one-piece, over-molded component. In some embodiments, a portion of the strain relief 18 is extruded, injection molded, or over molded while other portion(s) of the strain relief 18 are made using a different process.

As shown in FIG. 1, a first connector 42 may be disposed at the proximal end 22 of the strain relief 18. The first connector 42 includes an inner surface 44 and an outer surface 46. The first connector 42 may be threaded, for example, where threads are located on the first connector's 42 inner surface 44. The first connector 42 may be a female luer connector or a male luer connector. In some embodiments, at least a portion of the outer surface 33 of the strain relief 18 body member 28 covers at least a portion of the first connector 42. In some embodiments, at least a portion of the outer surface 33 of the strain relief 18 body member 28 covers the entire outer surface 46 of the first connector 42.

Any suitable connecting means may be used to couple the strain relief 18 to the handle 12. For example, as shown in FIG. 1, a medical device 2 may also include a second connector 48. A component extending through the body member 28, for example a medical device shaft 6 and/or tube 38, may also extend through or into the second connector 48. The second connector 48 may be coupled to the first connector 42. The second connector 48 may be complementary to the first connector 42 such that the first connector 42 and the second connector 48 may be coupled together. For example, the first connector 42 may be a threaded female luer connector and the second connector 48 may be a threaded male luer connector. In the example with the threaded female and male luer connectors, the connectors may be screwed together to couple (e.g., FIG. 9). A second connector 48 may be coupled to a handle 12 or formed integrally with a handle 12.

In some embodiments, the strain relief 18 may be made from a soft to semi-rigid material such as a plastic, polyurethane, PEBAX, polyethylene, polypropylene, fluorocarbon polymers, silicone, latex, polyvinyl chloride, cope loop tubing, like biocompatible polymeric materials, or a combination thereof. Any suitable material may be used to form the strain relief 18 such that the strain relief 18 is sufficiently flexible to facilitate maneuvering a tool 4 disposed inside a patient's body, but also has enough strength to provide support and prevent kinking or separation. The first and/or second connector 42, 48 may each be made from a material less flexible than the material used to make the strain relief 18. In some embodiments, a connector 42, 48 may be made from a semi-rigid to rigid plastic material such as a plastic, acrylonitrile butadiene styrene, like biocompatible polymeric materials, or a combination thereof.

Before using a strain relief 18 to direct a tool 4 of the medical device shaft 6 into a patient, in some embodiments, the strain relief 18 may be already provided on and stored with the medical device shaft 6, such that the strain relief 18 is in readiness for use by a physician. In some embodiments, the strain relief 18 may be stored separately from the medical device 2 and thus a physician places the strain relief 18 onto the medical device shaft 6 of the medical device 2 prior to use. To prep the strain relief 18 for use, the physician may thread either the distal end or proximal end 20, 22 of the strain relief 18 onto a medical device shaft 6.

A user may place the tool 4 inside a patient's body (which may be inserted with or without the use of a guide wire) such that the medical device shaft 6 may be used to direct the tool 4 to the desired location where an object to be extracted, or a clinical area to be investigated, observed, or interacted with is located. The user may manipulate the medical device 2 to perform a medical procedure without unnecessary medical device shaft 6 kinking or damage to a junction between the medical device shaft 6 and a handle 12.

After use, the user may decouple the strain relief 18 from medical device 2 such that the strain relief 18 may again be used for a subsequent procedure. The user may also remove (e.g., by peeling off) the strain relief 18 from the medical device shaft 6 as needed or desired. In some embodiments, the strain relief 18 may be configured to be peeled away from the medical device shaft 6, for example, for disposal. The strain relief 18 may include a slit or spiral cut on the body member 28 such that the strain relief 18 may be removed from the medical device 2 after the strain relief 18 has been used to direct the distal end 8 of the medical device shaft 6 to a desired location. Alternatively, the strain relief 18 may be disposed of subsequent to a procedure along with the medical device 2, functioning as a one-time-use device.

While various embodiments are described herein, the disclosure is not to be restricted except in light of the attached claims and equivalents. One skilled in the relevant art will recognize that numerous variations and modifications may be made to the embodiments described above, expressly including that the construction materials identified may be used in all embodiments, the relative and absolute sizes of component structures including connectors, lumens, depressions (including any combination of shapes and/or patterns of depressions), and/or inner tube(s) may be incorporated in any physically-possible combination in all embodiments and alternative embodiments encompassed by the claims. Moreover, the advantages described herein are not necessarily the only advantages and it is not necessarily expected that every embodiment will achieve all of the advantages described.

We claim:

1. A strain relief member for a medical device delivery system, comprising:
a body member comprising a first end comprising a first outer diameter, a second end comprising a second outer diameter, an inner surface facing a lumen that extends axially through the body member along a longitudinal axis, and an outer surface opposite to the inner surface,
wherein the outer surface of the body member comprises a plurality of depressions; and
a threaded first connector disposed at the first end of the body member,
wherein the body member comprises a first material and the first connector comprises a second material, and the first material is more flexible than the second material, and wherein a portion of the outer surface of the body member covers at least a portion of the first connector.

2. The strain relief member of claim 1, wherein the first outer diameter is larger than the second outer diameter.

3. The strain relief member of claim 1, wherein the first connector is a female luer connector or a male luer connector.

4. The strain relief member of claim 1, wherein the lumen comprises a diameter of about 1.0 French to about 4.5 French.

5. The strain relief member of claim 1, wherein a portion of the outer surface of the body members covers the first connector.

6. The strain relief member of claim 1, wherein the depressions are perpendicular to the longitudinal axis.

7. The strain relief member of claim 1, wherein the depressions comprise a line shape, a circle shape, an oval shape, a coil shape, or a combination thereof.

8. The strain relief member of claim 1, wherein the first material comprises polyurethane, silicone, latex, polyvinyl chloride, cope loop tubing, or any combination thereof.

9. The strain relief member of claim 1, wherein the second material comprises acrylonitrile butadiene styrene.

10. The strain relief member of claim 1, further comprising an inner tube, wherein at least a portion of the inner tube is within the lumen of the body member.

11. A strain relief member for a medical device delivery system, comprising:
a body member comprising a first end comprising a first outer diameter, a second end comprising a second outer diameter, an inner surface facing a lumen that extends axially through the body member along a longitudinal axis, and an outer surface opposite to the inner surface,
wherein the outer surface of the body member comprises a plurality of depressions; and
a first connector disposed at the first end of the body member,
wherein the body member comprises a first material and the first connector comprises a second material, and the first material is more flexible than the second material; and
a medical device shaft through at least a portion of the lumen of the body member.

12. The strain relief member of claim 11, wherein the first connector is threaded.

13. The strain relief member of claim 11, further comprising a second connector, wherein the second connector is coupled to the first connector.

14. The strain relief member of claim 13, wherein the second connector is complementary to the first connector.

15. The strain relief member of claim 11, wherein a portion of the outer surface of the body member covers at least a portion of the first connector.

16. The strain relief member of claim 13, further comprising a handle coupled to the second connector.

17. The strain relief member of claim 16, wherein the handle is a medical device handle.

18. The strain relief member of claim 11, further comprising a medical tool coupled to the medical device shaft.

19. The strain relief member of claim 18, wherein the medical tool is a basket, a snare, or a combination thereof.

20. A method of manufacturing a strain relief member for a medical device delivery system, comprising:
injection molding a strain relief member,
wherein the strain relief member comprises a body member comprising a first end comprising a first outer diameter, a second end comprising a second outer diameter, an inner surface facing a lumen that extends axially through the body member along a longitudinal axis, and an outer surface opposite to the inner surface,
wherein the outer surface of the body member comprises a plurality of depressions; and
a threaded first connector disposed at the first end of the body member; or
injection molding a threaded first connector, and over molding a strain relief member,
wherein the strain relief member comprises a body member comprising a first end comprising a first outer diameter, a second end comprising a second outer diameter, an inner surface facing a lumen that extends axially through the body member along a longitudinal axis, and an outer surface opposite to the inner surface,
wherein the outer surface of the body member comprises a plurality of depressions; and
the threaded first connector is disposed at the first end of the body member,
and
wherein the body member comprises a first material and the first connector comprises a second material, and the first material is more flexible than the second material, and wherein a portion of the outer surface of the body member covers at least a portion of the first connector, and
wherein a portion of the outer surface of the body member covers at least a portion of the first connector.
